(19) United States
(12) Patent Application Publication
(10) Pub. No.: US 2024/0122962 A1
(43) Pub. Date: Apr. 18, 2024

(54) PHARMACEUTICAL COMPOSITION FOR USE IN THE PREVENTION OR TREATMENT OF A CONDITION ASSOCIATED WITH A REDUCTION IN THE NUMBER OF LACTOBACILLI IN THE VAGINA

(71) Applicants: UCL Business Ltd, London (GB); King’s College London, London (GB)

(72) Inventors: Paul Long, London (GB); Alastair Sutcliffe, London (GB); Catherine Tuleu, London (GB)

(21) Appl. No.: 18/253,283

(22) PCT Filed: Nov. 27, 2021
(86) PCT No.: PCT/GB2021/052973

§ 371 Date: May 17, 2023

(30) Foreign Application Priority Data
Nov. 17, 2020 (GB) 2018068.3

(51) Int. Cl. A61K 31/715; A61K 9/00; A61K 9/06; A61K 45/06; A61K 47/38; A61P 15/02; A61P 31/04

(52) U.S. Cl. CPC A61K 31/715; A61K 9/0036; A61K 9/06; A61K 45/06; A61K 47/38; A61P 15/02; A61P 31/04

(57) ABSTRACT

The present invention provides a pharmaceutical composition for use in the prevention or treatment of a condition associated with a reduction in the number of Lactobacilli in the vagina of a subject relative to a healthy subject, wherein the composition comprises from 1.3 to 1.7 w/v % glycogen and less than 0.1 w/v % lactic acid.

### FIELD OF THE INVENTION

The present invention relates to a pharmaceutical composition comprising glycogen for use in preventing or treating conditions associated with a reduction in the number of Lactobacilli in the vagina of a subject relative to a healthy subject, for example vaginal infection or vaginal inflammation such as bacterial vaginosis, vaginitis, vulvovaginal candidiasis or Group B Streptococcus infection, or a method for the prevention or treatment of such a condition or resultant sequalae thereof with the composition.

### BACKGROUND TO THE INVENTION

Bacterial vaginosis (BV) is the most common vaginal infection in women of reproductive age worldwide, and is associated with significant adverse consequences including an increased risk of late miscarriage or preterm birth (1, 2), post-partum endometritis (3) and also a greater chance of acquiring sexually transmitted diseases, such as HIV (4). Reported prevalence rates range from 10-40% at any one time, depending upon the population studied. However, suboptimal methods of diagnosis and a high percentage of asymptomatic patients suggest the true prevalence of BV maybe very much higher (5). BV is associated with an increased volume of vaginal discharge having a foul, fishy odor.

The exact cause of BV is still unknown in the vast majority of instances, but alterations of both local host immunity and the genital tract microflora appear to contribute to the pathogenesis of BV (6). Under normal conditions, Lactobacillus bacteria are predominant in the vagina and are believed to control the growth of other microorganisms by producing hydrogen peroxide and lactic acid from vaginal glycogen to maintain vaginal acidity between pH 4 and 5. Lactobacillus is a genus of Gram-positive, facultative anaerobic or microaerophilic, rod-shaped, non-spore-forming bacteria.

In women with BV, however, normal vaginal lactobacilli are replaced by an overgrowth of other anaerobes with a concomitant decrease in lactobacilli numbers. Recent data suggest a primary role for Gardnerella vaginalis as a specific and perhaps sexually transmitted aetiological agent in BV (7-9), as was initially postulated by Gardner and Dukes in 1955 (10). Thus, while acid producing lactobacilli can be found in women with BV (11), their numbers may not be sufficient to overcome Gardnerella. vaginalis, which in turn replaces lactobacilli as the dominant microflora as the vaginal pH increases to around 7.8-8.2 (12). Conversely, numerous studies have shown that even when there is not a significant reduction in lactobacilli numbers, small decreases can be sufficient to allow overgrowth by yeast such as species of Candida, which cause a drop in pH below 4 consistent with symptom of vulvovaginal candidiasis ((27)-(32)).

Often women who experience the symptoms of BV complain of a foul, fishy odour and excessive vaginal discharge sufficiently unpleasant, that these women seek medical treatment. Traditional treatments have classically centred on the prescription-only antibiotics metronidazole and clindamycin. Metronidazole as a 7-day oral treatment has an 80-90% cure rate after 1 month. Side effects include nausea, abdominal cramps and a metallic taste. The patient must refrain from alcohol intake, as it may produce antabuse effects. It is not recommended in the first trimester of pregnancy. Metronidazole is also available in vaginal gels, for example, METROGEL VAGINAL®, yet despite their common use, vaginal gels are less than ideal. To be effective, the gels must be applied once or twice a day for a period of five days, usually at night. Clindamycin as a 7-day oral treatment has equal effects as metronidazole, and its side effects are less, although diarrhoea is possible and concerns about Clostridium difficile colitis have prevented widespread use (13, 14). Despite a high cure rate, a significant proportion of women suffer relapses and recurrences of BV, regardless whether the proceeding treatment was oral or intravaginal (15, 16). For example, a double-blind, placebo-controlled crossover trial has shown that intravaginal treatment with a 0.75% (w/v) metronidazole gel resulted in a recurrence rate of about 15% one month following treatment (17).

Antibiotics are a temporary treatment for BV. Antibiotics can eliminate the bacteria that cause BV, but at the same time also disrupt the natural balance of bacterial flora in the vagina, which can be disruptive in the long term leading to other infections including candidiasis.

Canesbalance® and Balance Active are commercial products which claim to reduce the symptoms of BV. These formulations are composed of lactic acid and glycogen. Because these formulations are acidic, they can cause irritation and even bleeding. The acidity of these formulations is to affect a rapid drop in the vaginal pH, reducing overgrowth of pathogenic bacteria and to promote regrowth of acid tolerant lactobacilli. These formulations, therefore, are not a cure and only offer at best, some temporary relief in some women. This regrowth will be only of the most acid tolerant and fast growing lactobacilli serotypes (perhaps dominated by a single or just a few serotypes), which may not persist overtime, resulting in replacement with the pathogenic overgrowth and recurrence of symptoms.

Many healthy women consider the symptoms of BV to indicate a lack of proper hygiene, rather than a medical problem and often use douches purchased without a prescription, rather than seek medical advice. The idea of washing out the foul smelling discharge with an acidic douche may have a simplistic appeal. Medically, however, douching is discouraged as studies have demonstrated an association between douching and Pelvic Inflammatory Disease (PID), ectopic pregnancy, tubal infertility, and reduced fertility (18-21). Different alternative measures have also been advised, including live yoghurt or Lactobacillus acidophilus preparations, although studies to date have not yet demonstrated benefits from the use of probiotics (15). In U.S. Pat. No. 6,440,949 the use of one or more saccharides (but not glycogen) in acidic prebiotic formulations is suggested as a method to decrease vaginal pH. In a Danish based clinical trial, the patent tested different concentrations of saccharides, but none of the concentrations showed a significant decrease in pH and, furthermore, none of the concentrations eliminated odor causing bacteria.

EP1072269B1 discloses the use of glycogen (alone at a concentration of between 2.5% and 17 w/v % and together with other polysaccharides) in a lactic acid gel as a treatment for BV; although clinical benefits were reported to be poor.

Therefore, in view of the fact that BV is currently the most prevalent form of vaginal infection in women of reproductive age, there is a real and immediate need for new compositions that address the shortcomings of currently available BV treatments. For example, it would be desirable to have available an intravaginal treatment that reduces the rate of recurrence of BV following a successful course of treatment; with additional benefits of reducing gynaecological complications such as preterm delivery. There is also a need to provide new treatments for other vaginal infections and inflammation.

### SUMMARY OF THE INVENTION

In an aspect of the invention, provided is a pharmaceutical composition for use in the prevention or treatment of a condition associated with a reduction in the number of Lactobacilli in the vagina of a subject relative to a healthy subject, wherein the composition comprises from 1.3 to 1.7 w/v % glycogen and less than 0.1 w/v % lactic acid.

The inventors have surprisingly found that the composition of the invention causes a significant increase in the number of Lactobacilli in the vagina of a treated subject, leading to the effective treatment of a number of vaginal infections and inflammations, for example bacterial vaginosis, vaginitis, vulvovaginal candidiasis or Group B Streptococcus.

In a particularly preferred embodiment the composition contains no lactic acid, i.e. the composition contains 0 w/v % lactic acid.

In a preferred embodiment the pharmaceutical composition further comprises water and a cellulose derivative, optionally from 94 to 97 w/v % water and from 2 to 5 w/v % of a cellulose derivative. Typically the pharmaceutical composition comprises from 1.3 to 1.7 w/v % glycogen, optionally from 1.4 to 1.6 w/v % glycogen, water and a cellulose derivative. Typically the pharmaceutical composition comprises from 1.3 to 1.7 w/v % glycogen, optionally from 1.4 to 1.6 w/v % glycogen, and an aqueous hydrogel comprising a cellulose derivative.

In a particularly preferred embodiment the pharmaceutical composition comprises about 1.5 w/v % glycogen.

Providing the correct amount of glycogen in the pharmaceutical composition is particularly important to achieve the therapeutic effect.

Without being bound by theory it is understood that a pharmaceutical composition comprising 1.3 to 1.7 w/v %, preferably 1.4 to 1.6 w/v %, most preferably about 1.5 w/v % glycogen, provides an optimum concentration of glycogen such that it acts as a carbon source for Lactobacilli, aiding in their growth. At lower concentrations, for example 1 w/v % glycogen, the concentration of carbon is not sufficient to support good Lactobacillus growth. Higher concentrations, for example 2 w/v % glycogen, or 2.5 to 17 w/v % as disclosed in EP1072269B1 can actually inhibit bacterial growth. The inhibition of bacterial growth at high saccharide concentrations is well known, and saccharides can be used as preservatives, for example in jams.

Typically, the glycogen is obtainable from an animal source, optionally wherein the glycogen is oyster, mussel or bovine glycogen.

Typically the cellulose derivative is a cellulose ether. The cellulose ether may be methylcellulose (MC), hydroxypropyl methylcellulose (HPMC), ethyl cellulose (EC), hydroxyethyl cellulose (HEC), sodium carboxymethylcellulose (NaCMC), or combinations thereof. Preferably the cellulose ether is hydroxypropyl methylcellulose.

The pH of the pharmaceutical composition may be from 5.7 to 6.7. Preferably the pH of the pharmaceutical composition is from 6.0 to 6.2.

Typically the pharmaceutical product is in the form of a hydrogel.

The pharmaceutical composition may be a vaginal pessary.

The pharmaceutical composition may be mucoadhesive, for example it may comprise a mucoadhesive film or gel or mucoadhesive in situ gelling liquid crystalline precursor system.

The pharmaceutical composition may further comprise one or more pharmaceutically acceptable excipients.

The pharmaceutical composition may further comprise one or more additional therapeutic agents. The one or more additional therapeutic agents may be selected from an antibiotic, an antibacterial agent, an antifungal agent, an antiparasitic agent, an antiviral agent, an antiseptic agent and an anti-inflammatory agent.

In a further aspect, the present invention provides a method for the prevention or treatment of a condition associated with a reduction in the number of Lactobacilli in the vagina of a subject relative to a healthy subject, the method comprising the step of administering to a subject in need thereof a pharmaceutical composition as defined above.

In an embodiment of the pharmaceutical composition for use or method of the invention the use or method comprises providing an increase in the number of Lactobacilli in the vagina of the subject relative to an untreated subject with the condition. In an embodiment of the pharmaceutical composition for use or method of the invention the use or method comprises restoring the number of Lactobacilli in the vagina to a normal level i.e. the individualized nature of the vaginal microbiome makes the response to the pharmaceutical composition subject-specific affording each patient their own ‘Personalized microbiome therapy’. The Lactobacilli may be endogenous Lactobacilli, i.e. normally present in the vagina of a subject who is healthy. The Lactobacilli may comprise Lactobacillus gasseri, Lactobacillus acidophilus, Lactobacillus paracasei, Lactobacillus salivarius, Lactobacillus johnsonii or combinations thereof. Fermentation of glycogen as a sole carbon source by Lactobacilli not only provides nutrition allowing restoration of Lactobacilli to a normal level, but the end products of the fermentation (lactic acid, hydrogen peroxide) cause a restoration in pH. In the case of bacterial vaginosis, the pH drops from alkaline to slightly acidic. Lactic acid has been widely demonstrated to inhibit growth of Candida species that cause vulvovaginal candidiasis. Therefore, a restoration in Lactobacilli numbers increases the concentration of lactic acid in the vagina, inhibiting the growth of pathogenic Candida species (33).

The condition to be prevented or treated is typically a vaginal infection, vaginal inflammation, or complications therefrom, optionally a recurrent vaginal infection or vaginal inflammation. The complication may be preterm birth, stillbirth, postpartum vaginal infection or vaginal inflammation.

In preferred embodiments of the invention the condition to be prevented or treated is bacterial vaginosis, vaginitis, vulvovaginal candidiasis or Group B Streptococcus infection.

In one embodiment the subject to be treated is pregnant.

In a preferred embodiment the condition to be prevented or treated is Group B Streptococcus infection and the subject is pregnant.

In embodiments of the composition for use or method of the invention the condition to be prevented or treated is associated with an increase in the number of pathogenic bacteria or yeast in the vagina of a subject relative to a healthy subject, optionally Gardnella vaginalis or Group B Streptococcus bacteria or a species of Candida yeast, optionally Candida albicans.

Typically the pharmaceutical composition is suitable for intravaginal administration.

### DESCRIPTION OF THE FIGURES

FIG. 1 shows average colony counts for a range of glycogen concentrations from Patient 1. Eight colonies from the patient were each grown in a medium comprising 0% w/v glycogen for 72 hours. 10 microlitres of this solution was then incubated for 96 hours in each glycogen concentration and subsequently plated onto Lactobacillus selective agar in triplicate n=24. Error bars show the deviation from the true value (mean) with samples showing significance marked with an asterisk (*).

FIG. 2 shows average colony counts for a range of glycogen concentrations from Patient 2. Three colonies from the patient were each grown in a medium comprising 0% w/v glycogen for 72 hours. 10 microlitres of this solution was then incubated for 72 hours in each glycogen concentration and subsequently plated onto Lactobacillus selective agar in triplicate n=9.

FIG. 3 shows average colony counts for a range of glycogen concentrations from Patient 3. Three colonies from the patient were each grown in a medium comprising 0% w/v glycogen for 72 hours. 10 microlitres of this solution was then incubated for 72 hours in each glycogen concentration and subsequently plated onto Lactobacillus selective agar in triplicate n=9.

FIG. 4 shows a pie chart of the relative proportions of bacterial species in the vagina of a patient (A) before treatment with the composition of the invention and (B) after treatment with the composition of the invention as determined by 16 S rRNA amplicon sequencing.

### DETAILED DESCRIPTION

In an aspect of the invention, provided is a pharmaceutical composition for use in the prevention or treatment of a condition associated with a reduction in the number of Lactobacilli in the vagina of a subject relative to a healthy subject, wherein the composition comprises from 1.3 to 1.7 w/v % glycogen and less than 0.1 w/v % lactic acid.

A skilled person would be aware of a number of conditions associated with a reduction in the number of Lactobacilli in the vagina of a subject relative to a healthy subject, for example vaginal infection or vaginal inflammation, in particular bacterial vaginosis, vaginitis, or Group B Streptococcus infection.

A skilled person would also know how to measure the number of Lactobacilli in the vagina of a subject. For example, a swab may be taken from the vagina of the subject, and used to inoculate a medium suitable for growth of Lactobacilli. After incubation, the grown bacteria are then cultured onto Lactobacilli specific agar plates, and incubated anaerobically at 37° C. for a suitable period of time. The number of bacterial colonies is then counted. The number of Lactobacilli is typically expressed in colony-forming units (CFU). Lactobacilli are the most prevalent and often numerically dominant microorganisms in the vagina, at 10⁷-10⁸ CFU mL⁻¹ of vaginal fluid in healthy premenopausal women (see Farage M A, Miller K W, Sobel J D (2010) Dynamics of the vaginal ecosystem-hormonal influences. Infect Dis Res Treat 3:1-15, and Boris S, Barbés C (2000) Role played by lactobacilli in controlling the population of vaginal pathogens. Microbes Infect 2:543-546). CFU is a unit used to estimate the number of viable bacteria in a sample. Viable is defined as the ability to multiply via binary fission under the controlled conditions. Counting with colony-forming units requires culturing the microbes and counts only viable cells, in contrast with microscopic examination which counts all cells, living or dead. The visual appearance of a colony in a cell culture requires significant growth, and when counting colonies it is uncertain if the colony arose from one cell or a group of cells. Expressing results as colony-forming units reflects this uncertainty. Typically, the pharmaceutical composition of the invention treats vaginal conditions wherein the CFU for vaginal Lactobacilli is significantly reduced relative to healthy subjects. The pharmaceutical composition of the invention causes a significant increase in the number vaginal Lactobacilli, i.e. a significant increase in the Lactobacilli CFU relative to untreated subjects suffering from the vaginal condition.

Thus in a preferred embodiment the present invention relates to a pharmaceutical composition for use in the prevention or treatment of vaginal infection or vaginal inflammation, optionally bacterial vaginosis, vaginitis, or Group B Streptococcus infection, wherein the composition comprises from 1.3 to 1.7 w/v % glycogen and less than 0.1 w/v % lactic acid.

The inventors have surprisingly found that the composition of the invention causes a significant increase in the number of Lactobacilli in the vagina of a treated subject, leading to the effective treatment of a number of vaginal infections and inflammations, for example bacterial vaginosis or vaginitis. It is understood that Lactobacilli on the vaginal epithelial surface use glycogen as a food source, producing lactic acid which keeps the environment at a lower pH and deters other bacterial types. The glycogen content of human vaginal tissue is discussed in Gregoire, A. T. et al, Fertility and Sterility, Vol. 22, No. 1, January 1971. However, whilst compositions comprising glycogen are disclosed in the prior art for the treatment of vaginal infections such as BV by altering the microflora of the vagina, glycogen is typically used in combination with another active ingredient such as lactic acid or another saccharide, or glycogen is used at a significantly higher concentration.

Providing the correct amount of glycogen in the pharmaceutical composition is particularly important to achieve the therapeutic effect.

Without being bound by theory it is understood that a pharmaceutical composition comprising 1.3 to 1.7 w/v %, preferably 1.4 to 1.6 w/v %, most preferably about 1.5 w/v % glycogen, provides an optimum concentration of glycogen such that it acts as a carbon source for Lactobacilli, aiding in their growth. At lower concentrations, for example 1 w/v % glycogen, the concentration of carbon is not sufficient to support good Lactobacillus growth. Higher concentrations, e.g. 2 w/v % glycogen, or 2.5 to 17 w/v % as disclosed in EP1072269B1 can actually inhibit bacterial growth. The inhibition of bacterial growth at high saccharide concentrations is well known, and saccharides can be used as preservatives, for example in jams.

Typically, the glycogen is obtainable from an animal source, optionally wherein the glycogen is oyster, mussel or bovine glycogen.

In a preferred embodiment the pharmaceutical composition further comprises water and a cellulose derivative, optionally from 94 to 97 w/v % water and from 2 to 5 w/v % of a cellulose derivative.

Typically the pharmaceutical composition comprises or consists of from 1.3 to 1.7 w/v % glycogen, optionally 1.4 to 1.6 w/v % glycogen, water and a cellulose derivative. For example, preferably the pharmaceutical composition comprises or consists of from 1.3 to 1.7 w/v % glycogen, optionally 1.4 to 1.6 w/v % glycogen, and an aqueous hydrogel comprising a cellulose derivative.

In a particularly preferred embodiment the pharmaceutical composition comprises about 1.5 w/v % glycogen.

A key advantage of the present invention is that the pharmaceutical composition can be formed, and can achieve a therapeutic effect using only three key components: glycogen, a cellulose derivative and water, although of course other components may be present. This simple composition is likely to provide fewer side effects, for example irritation and bleeding, than the compositions of the prior art.

The pharmaceutical composition comprises less than 0.1 w/v % lactic acid, optionally less than 0.05 w/v % lactic acid. The composition may contain trace amounts of lactic acid, but in the most preferred embodiment the composition contains no lactic acid, i.e. it contains 0 w/v % lactic acid. Prior art compositions comprising lactic acid for treating BV are known, for example compositions described in EP1072269B1, and commercially available Canesbalance® and Balance Active® formulations. Lactic acid is included in these formulations to decrease the pH of the composition. When administered to patients these formulations affect a rapid drop in vaginal pH, reducing overgrowth of pathogenic bacteria and promoting regrowth of acid tolerant Lactobacilli. However, these highly acidic lactic acid-containing compositions can cause irritation and bleeding in patients. In addition, the lactic-acid containing compositions typically only enable regrowth of the most acid tolerant and fast growing Lactobacilli serotypes, which may not persist over time, resulting in replacement with pathogenic bacteria and recurrence of symptoms.

The composition of the invention is not highly acidic, and does not comprise more than trace quantities of lactic acid, and preferably is free from lactic acid, avoiding irritation and bleeding caused by the acidic formulations of the prior art.

In addition, the pharmaceutical composition of the invention has superior stability and shelf life than Lactobacillus preparations of the prior art.

The glycogen may be obtainable from a marine organism, for example an oyster. The glycogen may be Type II glycogen. Glycogen from a marine organism source is commercially available, for example Type II glycogen from oyster is available from Sigma Aldrich, catalogue number G8751. The glycogen may also be from other marine (such as varieties of mussels) or terrestrial animal sources (including mammalian muscle or liver), but not a plant source. Glycogen is a multi-branched polysaccharide of glucose that serves as a form of energy storage in bacteria (as well as in humans, animals, and fungi). Thus it is understood that the glycogen provided in the compositions of the invention provide an energy or carbon source for Lactobacilli, enabling them to thrive. Glycogen is a branched biopolymer consisting of linear chains of glucose residues with an average chain length of approximately 8-12 glucose units. Glucose units are linked together linearly by α(1→4) glycosidic bonds from one glucose to the next. Branches are linked to the chains from which they are branching off by α(1→6) glycosidic bonds between the first glucose of the new branch and a glucose on the stem chain.

The cellulose derivative may be any polymer comprising cellulose. Typically the cellulose derivative is a cellulose ether. The cellulose ether may be methylcellulose (MC), hydroxypropyl methylcellulose (HPMC), ethyl cellulose (EC), hydroxyethyl cellulose (HEC), sodium carboxymethylcellulose (NaCMC), or combinations thereof. Preferably the cellulose ether is hydroxypropyl methylcellulose. A combination of hydroxypropyl methyl cellulose and methylcellulose may also be used. Cellulose ethers are commercially available, for example Metalose® 90SH-400 from Shin Etsu. The cellulose derivative may also be cellulose sulfate (Ushercell).

The pH of the pharmaceutical composition may be from 5.7 to 6.7. The pH of the composition may be from 6.0 to 6.5 or from 6.0 to 6.3. Preferably the pH of the pharmaceutical composition is from 6.0 to 6.2.

Typically the pharmaceutical product is in the form of a hydrogel. Hydrogels comprise a cross-linked network of hydrophilic polymers, such as cellulose ethers. Hydrogels possess the ability to absorb a large amount of water and swell, while maintaining their three-dimensional structure.

The pharmaceutical composition may be a vaginal pessary, typically a vaginal hydrogel pessary. In alternative embodiments the pharmaceutical composition may be a tablet, liquid suspension or dispersion, dried powder, topical ointment, cream, foam, gel, polymeric nanoparticles, bioadhesive polymers, fast dissolving film, suppository, or aerosol. The pharmaceutical composition may be applied to a base material, for example a cotton or gauze material, such as a tampon.

The pharmaceutical composition may be administered directly to the vagina or can be administered using a solid device, for example an applicator.

The pharmaceutical composition may be mucoadhesive. Traditional commercial preparations for treating vaginal conditions are known to reside in the vaginal cavity for a relatively short period of time owing to the self-cleaning action of the vaginal tract, and often require multiple daily doses to ensure the desired therapeutic effect. The composition of the present invention is preferably mucoadhesive, i.e. it is capable of adhering to the vaginal mucosa. The mucoadhesive nature of the composition prolongs the residence time of the pharmaceutical composition in the vaginal cavity. Cellulose ethers, such as HPMC are typically mucoadhesive polymers.

The pharmaceutical composition for use of the invention may comprise of or consist of a mucoadhesive film, gel or mucoadhesive in situ gelling liquid crystalline precursor system.

The pharmaceutical composition for use may further comprise one or more pharmaceutically acceptable excipients. Pharmaceutically acceptable excipients that can be included in the pharmaceutical compositions of the present invention include, for example, physiologically tolerable surfactants, solvents, emollients, colorants, fragrances, gelling agents, emulsifiers, buffering agents, binders, antioxidants, disintegrants, suspending agent, chelating agent and the like, which are well known in the art. Possible excipients are set out in Table II of “Compendium of Pharmaceutical Excipients for Vaginal Formulations”, S. Garg et al, Pharmaceutical Technology Drug Delivery 2001.

The pharmaceutical composition may be free from preservatives, for example the composition may comprise less than 0.1 w/v % preservative.

The pharmaceutical composition may further comprise one or more additional therapeutic agents. The one or more additional therapeutic agents may be selected from an antibiotic, an antibacterial agent, an antifungal agent, an antiparasitic agent, an antiviral agent, an antiseptic agent and an anti-inflammatory agent.

The one or more additional therapeutic agents may be selected from:
antibacterial agents such as C31G, trimethoprim, sulfamethoxazole, and chloromycetin;
antiseptic agents such as chlorhexidine gluconate;
antibiotic agents such as erythromycin, penicillins, cephalosporins and their derivatives, ampicillin, methicillin, and doxycycline;
anti-inflammatory agents such as naproxen, indomethacin, and hydrocortisone;
antiparasitic agents such as thiabendazole;
antiprotozoal agents such as metronidazole, and chloroquine hydrochloride;
antiviral agents such as dextran sulfate and other sulfated polysaccharides, squalamine, and vidarabine;
and antifungal agents such as ketoconazole, flucytosine, itraconazole, amphotericin B, nystatin, butoconazole nitrate, and clotrimazole.

In a further aspect, the present invention provides a method for the prevention or treatment of a condition associated with a reduction in the number of Lactobacilli in the vagina of a subject relative to a healthy subject, the method comprising the step of administering to a subject in need thereof a pharmaceutical composition as defined above.

The subject may be a human or non-human animal.

In an embodiment of the pharmaceutical composition for use or method of the invention the use or method comprises providing an increase in the number of Lactobacilli in the vagina of the subject relative to an untreated subject with the condition. In one embodiment the increase in the number of Lactobacilli is achieved for a period of at least 48 hours, optionally 72 hrs. In an embodiment of the pharmaceutical composition for use or method of the invention the use or method comprises restoring the number of Lactobacilli in the vagina to a normal level, i.e. restoring the number of Lactobacilli in the vagina of the subject to a number of Lactobacilli found in a healthy subject. The number of Lactobacilli in a healthy subject is patient dependent, but a healthy premenopausal woman may have 10⁷-10⁸ CFU Lactobacilli mL⁻¹ vaginal fluid.

Methods of measuring the number of lactobacilli in the vagina are discussed above and would be known to a person skilled in the art. For example, a swab may be taken from the vagina of the subject, and used to inoculate a medium suitable for growth of Lactobacilli. After incubation, the grown bacteria are then cultured onto Lactobacilli specific plates, and incubated for a suitable period of time. The number of bacterial colonies is then counted. The number of Lactobacilli is typically expressed in colony-forming units (CFU). The Lactobacilli may be endogenous Lactobacilli, i.e. normally present in the vagina of a subject who is healthy. It will be understood that the vaginal microflora can vary significantly between individuals, and there may be significant variation in the number and type of Lactobacilli and other microorganisms present in the vaginal cavity between individuals. However, the Lactobacilli may comprise Lactobacillus gasseri, Lactobacillus acidophilus, Lactobacillus paracasei, Lactobacillus salivarius, Lactobacillus johnsonii or combinations thereof.

The condition to be prevented or treated is typically a vaginal infection, vaginal inflammation, or complications therefrom. The condition may be a recurrent vaginal infection or vaginal inflammation. A recurrent vaginal infection or inflammation is one in which symptoms recur despite initial reduction or elimination of symptoms, for example symptoms of infection or inflammation recur after one, two or three months. An infection may recur because although a population of pathogenic bacteria is significantly reduced after an initial treatment, allowing regrowth of the beneficial bacteria such as Lactobacilli, the pathogenic bacteria will not be completely eliminated and will regrow over time, causing a recurrence of the infection.

The composition of the invention is particularly suitable for treating recurrent infections because it is understood to support the regrowth of any Lactobacilli, not just those which are acid tolerant such as those found after treatment with the lactic-acid containing pharmaceutical compositions of the prior art. By supporting regrowth of a broader range of Lactobacilli, the regrowth of pathogenic bacteria or yeast is less likely, preventing recurrence of an infection such as BV or vulvovaginal candidiasis.

The pharmaceutical composition may be used to prevent or treat complications of the vaginal infection or vaginal inflammation such as preterm labour or birth, stillbirth, postpartum vaginal infection or vaginal inflammation. It is well known that vaginal infections such as BV provide an increased risk of preterm labour, birth, stillbirth, first-trimester miscarriage, optionally in women undergoing IVF, amniotic-fluid infection, chorioamnionitis, endometritis after childbirth or abortion, infections after hysterectomy, pelvic inflammatory disease, and postpartum infection/inflammation (see for example Romero, R. et al. (2001). “The role of infection in preterm labour and delivery,” Journal of Pediatric and Perinatal Epidemiology, 15(2), pp. 41-56., Warr A J, et al, “Sexually transmitted infections during pregnancy and subsequent risk of stillbirth and infant mortality in Kenya: a prospective study”, BMJ 2018; 0:1-7. doi:10.1136/sextrans-2018-053597, Baqui et al, “Prevalence of and risk factors for abnormal vaginal flora and its association with adverse pregnancy outcomes in a rural district in north-east Bangladesh”, Acta Obstet Gynecol Scand. 2018; 1-11, and Paavonen, J. et al, “Bacterial Vaginosis and Desquamative Inflammatory Vaginitis”, N Engl J Med 2018; 379:2246-2254).

In preferred embodiments of the invention the condition to be prevented or treated is bacterial vaginosis, vaginitis, vulvovaginal candidiasis or Group B Streptococcus infection.

In embodiments of the composition for use or method of the invention the condition to be prevented or treated is associated with an increase in the number of pathogenic bacteria, optionally Gardnella vaginalis bacteria or yeast in the vagina of a subject relative to a healthy subject. Pathogenic bacteria or yeast are any bacteria or yeast which can cause a disease or medical condition.

Typically the pharmaceutical composition is suitable for intravaginal administration. The pharmaceutical composition may be administered whilst the patient is supine, typically at night time.

In an additional aspect, the present invention provides a method of preparing a pharmaceutical composition, the method comprising:
(a) heating water to about 80° C.
(b) gradually adding a cellulose derivative to the heated water under agitation in an amount to provide an aqueous solution comprising 2 to 5 w/v % of the cellulose derivative
(c) cooling the aqueous solution to form a gel
(d) separately providing an aqueous solution of glycogen at 1 to 5° C.;
(e) mixing the gel with the aqueous solution of glycogen to provide the pharmaceutical composition, wherein the pharmaceutical composition comprises 1.3 to 1.7 w/v % glycogen, optionally 1.4 to 1.6 w/v %, optionally about 1.5 w/v %.

The cellulose derivative may be any cellulose derivative described above, for example HPMC. Agitation is typically achieved by stirring the solution, for example with a magnetic stirrer. In step (e) the mixing is very slow, or gradual mixing of the gel with the aqueous solution of glycogen.

The direct product of the method is a preferred embodiment of the composition used in the pharmaceutical composition for use or method of treatment of the invention.

The invention will now be further described by way of Examples, which are meant to serve to assist one of ordinary skill in the art in carrying out the invention and are not intended in any way to limit the scope of the invention.

### Example 1

This study was based on a within subject variation, therefore, only one patient was enrolled to the study after completion of screening questionnaires and giving their consent. The patient was diagnosed with BV using three of the Amsel's criteria (positive whiff test, grayish white homogeneous discharge and presence of clue cells). This method is well known to a person skilled in the art. A swab was taken from the patient and inoculated in artificial vaginal fluid (AVF) where the carbon source was replaced with glycogen.

Gel Formulation

The gel was prepared containing the following concentrations of glycogen, 0, 0.5, 1.0, 1.5 and 5% w/v in 100 ml glass media bottles. Both cold and hot processes of gel making were utilized in this method where 60 mL of water was heated to 80° C. to which 2.5 g of the semisynthetic polymer, hydroxypropyl methylcellulose (HPMC) (Metolose® 90SH-400) was gradually added whilst the solution was continually being stirred on a magnetic hot-plate stirrer. Glycogen was added to a 20 mL volumetric flask in the relative quantities and made up with cold water, also being placed on a magnetic stirrer. The formulated gel was then mixed and allowed to cool to room temperature. The dissolved glycogen was then added to the gel sample and the remaining 20 mL of cold water also added gradually. All samples were then labelled, autoclaved and placed in the fridge.

Bacterial Growth Media Preparation

3 L (144 agars) of the Rogosa Agar was prepared by adding 20.5 g of the growth media into 6×500 mL glass media bottles to which 1.3 mL of glacial acetic acid was added, before making it up to 250 mL with distilled water. The samples were then placed in the autoclave before being poured into the plates under sterile conditions.

Preparation of the Artificial Vaginal Fluid

Two Artificial Vaginal Fluids (AVFs) were prepared, one containing carbon (control) and one not. 300 mL of the AVF not containing carbon was prepared by the addition of the relative amounts of ingredients shown in table 1, and making it up to 300 mL with distilled water. The same procedure was carried out for the second AVF, but in 100 mL with the addition of the carbon amount stated in Table 1, before placing both fluids into the autoclave. Within a sterile fume hood, 10 mL of the control AVF was added to eight Sterilin specimen tubes, labelled and placed in the fridge. 5 mL of the second AVF was then also added to eight different tubes for each concentration of glycogen including the blank before adding 5 mL of the corresponding concentration of the prepared gels, labelling and placing in the fridge.

TABLE 1
Relative amounts of ingredients used to prepare both AVFs

| | Amount (g) | Amount (g) |
|---|---|---|
| Ingredient | per 300 mL | per 100 mL |
| Peptone | 1.5 | 0.5 |
| Yeast extract | 0.75 | 0.25 |
| Sodium acetate | 0.75 | 0.25 |
| Dipotassium phosphate | 0.3 | 0.1 |
| Ammonium sulphate | 0.3 | 0.1 |
| Magnesium sulphate | 0.03 | 0.01 |
| Carbon source | — | 1.5 |

Sub-Culturing Bacterial Samples

Swabs retrieved from patients positively diagnosed with BV by using three of the Amsel's criteria (positive whiff test, grayish white homogeneous discharge and presence of clue cells) were incubated in a broth containing a 1.5% w/v of glycogen and glucose for 3-4 days anaerobically at 37° C. to encourage the growth of the Lactobacilli bacterial strains. A 10 μL sample was then inoculated into three plates specific for Lactobacilli and incubated for a further 3-4 days anaerobically.

Sub-Culturing into Gel Samples, Plating and Colony Counting

Different colonies from the grown bacterial colonies were inoculated into each of the control broths containing carbon using a 10 μL inoculation loop and incubated for 3 days anaerobically. From the grown bacteria, 10 μL was then inoculated into all 5 gel sample broths including the blank and further incubated for 4 days. The grown bacteria were then cultured onto Lactobacilli specific plates set in triplicates for each gel concentration and incubated for 3 days anaerobically before being counted.

Bacterial Colony Counts

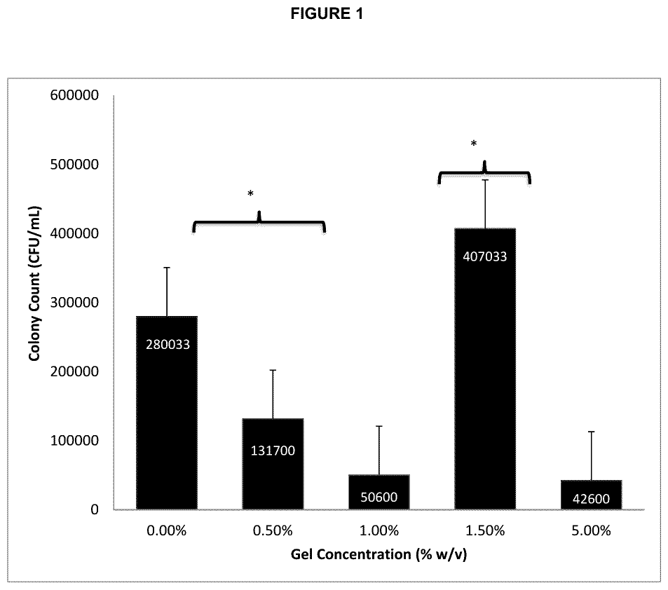

All grown bacterial colonies were counted on a Stuart SC6 colony counter. FIG. 1 shows the average of the bacterial counts for each gel glycogen concentration, attained from the eight colonies inoculated from the patient swabs. It is apparent that the glycogen concentration with the highest bacterial count is 1.5% w/v; this result is consistent with all eight colonies. The standard deviation error bars also show the deviation of each bacterial count from mean value (true value).

An analysis of variance (ANOVA) test was carried out to determine the deviation between the counts and whether the variable (gel concentration) has an effect on the bacterial counts or not. Where the f-value is greater than the f-critical value, the null hypothesis is rejected. The null hypothesis being that the variable has an effect on the bacterial colony count. When the null hypothesis is rejected, the alternative hypothesis must be accepted which is that the variable does not have an effect on the colony count. It was found that for colonies one, two, three and six, the f-critical value was lower than the f-value, rejecting the null hypothesis and accepting the alternative that the gel concentration does not have an effect on the colony count. On the other hand, colonies four, five, seven, and eight, all accepted the null hypothesis due to the f-critical value being higher than the f-value, suggesting there is an evident correlation between the variable and the colony count.

### Example 2

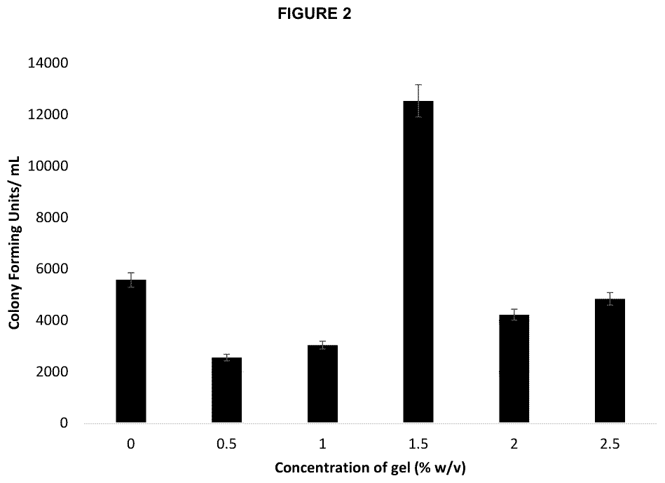

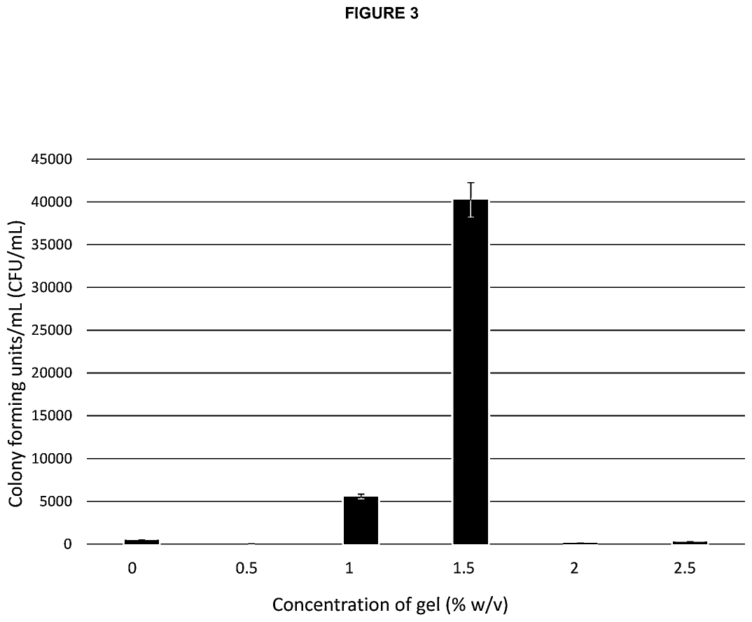

The study was repeated with 2 further patients, the results of which are shown in FIGS. 2 and 3. The same protocol was used for patients 2 and 3 as for patient 1, with the exception that three colonies rather than eight colonies from each patient were grown, and the inoculated control broth was incubated for a shorter period of time (2 days rather than 3 days). In addition, the formulations tested contained 0, 0.5, 1, 1.5. 2 and 2.5 w/v % glycogen. To summarise, different colonies from the grown bacterial colonies were inoculated into each of the control broths containing carbon using a 10 μL inoculation loop and incubated for 2 days (not 3 days as in Example 1) anaerobically. From the grown bacteria, 10 μL was then inoculated into all 5 gel sample broths including the blank and further incubated for 4 days. The grown bacteria were then cultured onto Lactobacilli specific plates set in triplicates for each gel concentration and incubated for 3-days anaerobically before being counted.

Examples 1 and 2 demonstrate that that the composition of the invention comprising glycogen has the ability to restore the vaginal flora, with the composition comprising 1.5% w/v glycogen achieving the highest counts of the bacterial species that typically dominates the vaginal environment in healthy women, Lactobacillus. The restoration of this bacterial species is associated with the treatment of BV and therefore compositions of the invention comprising from 1.3 to 1.7 w/v % glycogen, optimally 1.5 w/v % glycogen, can provide an efficacious non-antibiotic treatment of BV and other vaginal infections and inflammations. It can be seen from FIGS. 1 to 3 that compositions of the invention comprising 1.5 w/v % glycogen provide a much higher Lactobacillus count than compositions outside the scope of the claim comprising 0.5, 1.0, 2.0, 2.5 or 5.0 w/v % glycogen. Without being bound by theory it is understood that 1.5 w/v % glycogen provides an optimum concentration of glycogen such that it acts as a carbon source for Lactobacilli, aiding their growth. At lower concentrations, for example 1 w/v % glycogen, the concentration of carbon is not sufficient to support good Lactobacillus growth. Higher concentrations, e.g. 2 w/v % glycogen, or 2.5 to 17 w/v % as disclosed in EP1072269 can actually inhibit bacterial growth.

### Example 3—Comparison with Commercially Available Composition (Canesbalance® BV Gel)

Materials & Methods

Microbiological assessment of pessary performance in vitro: Two vaginal swabs were taken from opposite sides of the vagina of each patient. The two vaginal swabs were then inoculated into either 10 mL Vaginal Simulating Fluid or, 5 mL Vaginal Simulating Fluid where the carbon source had been replaced by 5 mL of the pessary composition of the invention (gel formulation of Example 1 comprising 1.5% w/v glycogen). Vaginal Simulating Fluid or Artificial Vaginal Fluid has been previously described in D. H. Owen and D. F. Katz, “A Vaginal Fluid Simulant”, Contraception, 1999; 59; 91-95, and M. S. J. Tomas and M. E. Nader-Macias, “Effect of a medium simulating vaginal fluid on the growth and expression of beneficial characteristics of potentially probiotic lactobacilli”, Communicating Current Research and Educational Topics and Trends in Applied Microbiology, 2007.

The Vaginal Simulating Fluid (or Artificial Vaginal Fluid) contained glucose (adjusted to a final concentration of 1.5%), peptone (10.0), yeast extract (5.0), sodium acetate (5.0), dipotassium phosphate (2.0), ammonium sulphate (2.0), magnesium sulphate (0.2), manganese sulphate (0.1), Tween 80 (1 mL), pH 4.5. Both broths were incubated anaerobically and without agitation at 37° C. for 72 hrs. The broths were then sub-cultured onto Lactobacillus Selection Agar (Rogosa agar). After 96 hrs anaerobic incubation at 37° C., the number of colonies visible on the agar plates was compared. Only lactobacilli can grow on the Rogosa agar and this was confirmed by light microscopy of Gram stained colonies, which gave morphology consistent with lactobacilli. The numbers of bacteria recovered after initial incubation in Vaginal Simulating Fluid will give an indication of the numbers of residual lactobacilli present during active BV disease. We were expecting that the numbers of bacteria recovered after initial incubation in Vaginal Simulating Fluid where the carbon source was replaced by the pessary, would show a shift towards increased numbers of Lactobacillus spp., if the glycogen was performing as a prebiotic in the pessary formulation.

Results

Table 2 below shows the number of bacteria recovered after 96 hours incubation on media specific for lactobacilli following pre-treatment of swabs for 72 hours in Artificial Vaginal Fluid, our formulation or the market brand leader Canesbalance®.

The Artificial Vaginal Fluid contains glucose as the sole carbon source and so should allow for all types of bacteria sampled from the patient to grow, including lactobacilli. Our formulation contains glycogen as the sole carbon source and should only encourage regrowth of the residual lactobacilli population, if this population still exists in the diseased vaginal flora. Canesbalance® contains glycogen (and lactic acid) and should, like our formulation; act as a prebiotic to encourage regrowth of residual lactobacilli.

The results show that there were significantly (p<0.05) more lactobacilli recovered following pre-incubation of the swabs in the glycogen only formulation of the invention compared to the Artificial Vaginal Fluid and, surprisingly, Canesbalance®. This suggests that the formulation of the invention can encourage significant regrowth of the lactobacilli that remain during a disease episode. The minor variation in the results between patients was as expected from a randomly selected patient population, suggesting that the majority of patients would respond in the same way if our formulation were used as a treatment.

TABLE 2

Mean number of lactobacilli
n = 3 (10⁴ × CFU mL⁻¹)

| Patient | Artificial | Glycogen only |
|---|---|---|
| number | Vaginal Fluid | formulation |
| 1 | 0.2 | 3.6 |
| 2 | 0.6 | 3.7 |
| 3 | 0 | 1.1 |
| 4 | 0 | 1.1 |
| Average | 0.2 | 2.4 |
| | | Glycogen only |
| | Canesbalance ® | formulation |
| 5 | 0 | 3.3 |
| 6 | 0 | 1.2 |
| 7 | 0 | 3.2 |
| 8 | 0 | 0 |
| 9 | 0 | 0.05 |
| 10 | 0 | 0 |
| 11 | 0 | 0 |
| 12 | 0 | 3.8 |
| 13 | 0 | 1.2 |
| 14 | 0 | 0 |
| 15 | 0 | 1.4 |
| 16 | 0 | 4.2 |
| 17 | 0 | 10.4 |
| 18 | 0 | 9 |
| Average | 0 | 2.7 |

### Example 4—Addition of Lactic Acid Inhibits Lactobacillus Growth

A gel formulation was prepared according to Example 1 comprising 1.5 w/v % glycogen. A comparative gel formulation was prepared according to Example 1 comprising 1.5 w/v % glycogen, but with the addition of lactic acid at a concentration of 225 mg per 5 mL. The comparative formulation had a pH of 3.5. The comparative formulation had the same lactic acid concentration and pH as commercial product Canesbalance®. Table 3 demonstrates that the addition of lactic acid to the formulation of the invention inhibits lactobacillus growth.

TABLE 3

Mean number of lactobacilli
n = 3 (10⁴ × CFU mL−¹)

| | Glycogen only | Glycogen only |
|---|---|---|
| Patient | formulation | formulation + |
| Number | (Invention) | lactic acid |
| 21 | 2.9 | 0.3 |
| 22 | 2.3 | 0 |
| 23 | 3.9 | 0 |

### Example 5—Bacterial Carbon Source

Swabs were incubated in either in Vaginal Simulating Fluid or Vaginal Simulating Fluid in combination with the gel formulation of the invention (1.5 w/v % glycogen), and broths were plated onto blood agar and incubated aerobically for 72 hrs at 37° C. (blood agar will encourage most bacteria, including BV associated bacteria; aerobic conditions were used because lactobacilli are unlikely to grow but any colonies showing signs of alpha haemolysis from were excluded from the counts as potential lactobacilli). If the bacterial counts were higher for the formulation of the invention than the control, this would suggest that other bacteria were using the formulation of the invention as a carbon source and out competing lactobacilli (i.e. not being inhibited by the lactobacilli). This was not the case, and Table 4 below shows that the mean number of bacteria was lower in the presence of the gel formulation of the invention than in the control, meaning that bacteria other than lactobacilli were not using the formulation of the invention as a carbon source.

TABLE 4

| | |
|---|---|
| | Mean |
| Blood agar | n = 3 (10⁴ × CFU mL⁻¹) |

| Patient | | Glycogen only formulation |
|---|---|---|
| number | Control | (invention) |
| 24 | 2.7 | 0.7 |
| 25 | 4.1 | 3.7 |

### Example 6—Physico-Chemical Parameters Indicative of Formulation of the Invention Encouraging Regrowth of Lactobacilli

Table 5 indicates that the administration of the gel formulation of the invention containing 1.5 w/v % glycogen leads to improvements in vaginal pH, lactic acid concentration and hydrogen peroxide concentration relative to administration of a control formulation.

TABLE 5

| | | | D-lactic acid | | Hydrogen peroxide | |
|---|---|---|---|---|---|---|
| | pH (>4.5 indicates | | concentration mM | | concentration μM | |
| | BV) | | (healthy~100 mM) | | (healthy~100 μM) | |
| | | Glycogen | | Glycogen | | Glycogen |
| | | only | | only | | only |
| Patient | | formulation | | formulation | | formulation |
| number | Control | (invention) | Control | (invention) | Control | (invention) |
| 24 | 5.5 | 4.2 | 0 | 45 | 0 | 0 |
| 25 | 5.2 | 3.8 | 22.5 | 90 | 0 | 88 |

### Example 7—Alteration of Vaginal Bacterial Population

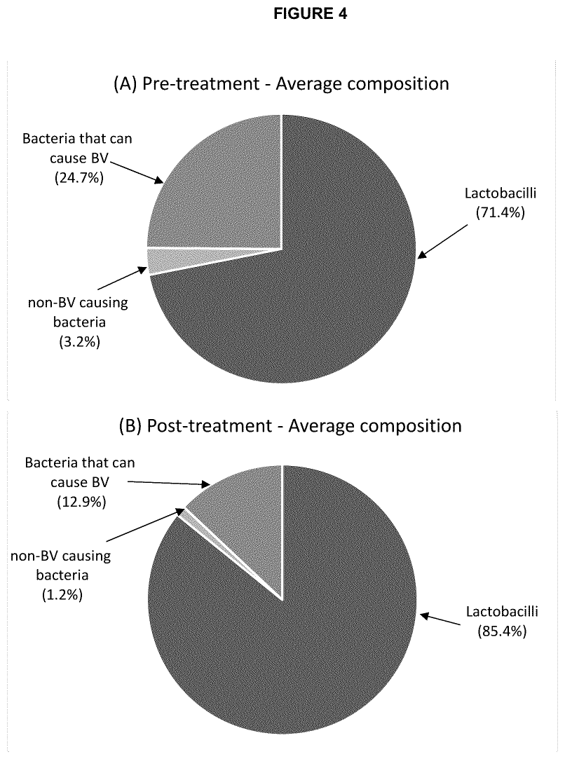

FIG. 4 shows a pie chart of the relative proportions of bacterial species in the vagina of a patient (A) before treatment with the composition of the invention and (B) after treatment with the composition of the invention as determined by amplicon sequencing the V1/V2 hypervariable region of bacterial 16 S rRNA gene (Ravel J, et al. Vaginal microbiome of reproductive-age women (Proc Natl Acad Sci USA. 2011; 108:4680-4687. doi: 10.1073/pnas.1002611107). It can be seen that the proportion of Lactobacilli increases from 71.4% (pre-treatment) to 85.4% (post-treatment). The proportion of Enterococci decreases from 24.7% (pre-treatment) to 12.9% (post-treatment).

### Example 8—Treatment of Group B Streptococcus Infection in Pregnant Patients

Pregnant patients known to be colonised by Group B streptococci were recruited. The method for detection of Group B Streptococci carriage followed UK Standards for Microbiology Investigations, Public Health England: Bacteriology B 58 Issue no: 3.1 Issue date: 26.06.18—as shown in Table 6 below.

Results of culture dependent colony counts of Group B streptococci (GBS) from swabs incubated in the LIM control broth vs LIM broth plus 1.5% w/v glycogen are provided in Table 7. Colonies were counted after incubation on chromogenic agar and results from 10 of the 12 patients show a significant (p≤0.05) reduction in numbers from swabs incubated in the formulation of the invention comprising 1.5% w/v glycogen, providing evidence that there would be a reduction in carriage of GBS in pregnant women if the formulation of the invention is used as a treatment.

TABLE 6

| Clinical | | | Incubation | | | | |
|---|---|---|---|---|---|---|---|
| details/ | | | Temp | | | Cultures | |
| Conditions | Specimen | Standard media | ° C. | Atmos | Time | read | Target organism(s) |
| Carriage of | Maternal low | LIM Broth (5 mL)^{†}: Todd-Hewitt broth | 35-37 | 5% CO₂ | 18- | N/A | |
| Group B | vaginal and | supplemented with 10 μg/mL colistin - or | | | 24 hr | | |
| streptococci | anorectal | 8 μg/mL gentamicin and 15 μg/mL nalidixic | | | | | |
| | swabs | acid | | | | | |
| | | Then subculture to: | 35-37 | 5% CO2 | 24- | 18-24 hr | Group B streptococci |
| | | Blood agar | | | 48 hr | and | |
| | | or | | | | 48 hr | |
| | | Selective agar | 35-37 | Ambient | 24- | 18-24 hr | |
| | | or | | | 48 hr | | |
| | | Chromogenic agar | 35-37 | Ambient | 24- | 18-24 hr | |
| | | | | | 48 hr | | |

^{†}The bottle should contain a volume of broth sufficient to cover the swabs

TABLE 7

Mean number of GBS colonies
n = 9 (10⁴ × CFU mL⁻¹)

| Patient No. | Control | Formulation of invention |
|---|---|---|
| 1 | 4.7 | 0.29 |
| 2 | 2.7 | 0.6 |
| 3 | 4.9 | 2.2 |
| 4 | 0.85 | 0.4 |
| 5 | 0.46 | 0.11 |
| 6 | 5.22 | 2.35 |
| 7 | 4.39 | 1.83 |
| 8 | 2.8 | 1.74 |
| 9 | 4.5 | 1.99 |
| 10 | 4.78 | 1.25 |
| 11 | 0.66 | 0.41 |
| 12 | 2.23 | 2.14 |

For each of patients 1 to 10, P < 0.05.

All publications mentioned in the above specification are herein incorporated by reference. Various modifications and variations of the described compositions for use and methods of the invention will be apparent to those skilled in the art without departing from the scope and spirit of the invention. Although the invention has been described in connection with specific preferred embodiments, it should be understood that the invention as claimed should not be unduly limited to such specific embodiments. Indeed, various modifications of the described modes for carrying out the invention which are obvious to those skilled in pharmaceutical science, medicine or related fields are intended to be within the scope of the following claims.

### REFERENCES

(1) Eschenbach D A, Gravett M G, Chen K C, Hoyme U B, Holmes K K. Bacterial vaginosis during pregnancy. An association with prematurity and postpartum complications. Scand J Urol Nephrol Suppl. 1984; 86:213-222.
(2) Hillier S L, Nugent R P, Eschenbach D A, et al. Association between bacterial vaginosis and preterm delivery of a low-birth-weight infant. The Vaginal Infections and Prematurity Study Group. N Engl J Med. 1995; 333(26):1737-1742.
(3) Watts D H, Eschenbach D A, Kenny G E. Early postpartum endometritis: the role of bacteria, genital mycoplasmas, and Chlamydia trachomatis. Obstet Gynecol. January 1989; 73(1):52-60.
(4) Martin H L, Richardson B A, Nyange P M, et al. Vaginal lactobacilli, microbial flora, and risk of human immunodeficiency virus type 1 and sexually transmitted disease acquisition. J Infect Dis. December 1999; 180(6):1863-1868.
(5) Koumans E H, Kendrick J S. Preventing adverse sequelae of bacterial vaginosis: a public health program and research agenda. Sex Transm Dis. May 2001; 28(5):292-297.
(6) Witkin, S. S., I. M. Linhares, P. Giraldo, and W. J. Ledger. 2007. An altered immunity hypothesis for the development of symptomatic bacterial vaginosis. Clin Infect Dis 44:554-7.
(7) Fredricks D N, Fiedler T L, Thomas K K, Mitchell C M, Marrazzo J M. Changes in Vaginal Bacterial Concentrations with Intravaginal Metronidazole Therapy for Bacterial Vaginosis as Assessed by Quantitative PCR. J Clin Microbiol. Jan. 14, 2009.
(8) Josey W E, Schwebke J R. The polymicrobial hypothesis of bacterial vaginosis causation: a reassessment. Int J STD AIDS. March 2008; 19(3):152-154.
(9) Schwebke J R, Rivers C, Lee J. Prevalence of Gardnerella vaginalis in Male Sexual Partners of Women With and Without Bacterial Vaginosis. Sex Transm Dis. Sep. 10, 2008.
(10) Gardner H L, Dukes C D. Haemophilus vaginalis vaginitis: a newly defined specific infection previously classified non-specific vaginitis. Am J Obstet Gynecol. May 1955; 69(5):962-976.
(11) Aroutcheva, A. A., J. A. Simoes, K. Behbakht, and S. Faro. 2001. Gardnerella vaginalis isolated from patients with bacterial vaginosis and from patients with healthy vaginal ecosystems. Clin Infect Dis 33:1022-7.
(12) Rosenstein, I. J., et al, Relationship between hydrogen peroxide producing strains of lactobacilli and vaginosis associated with bacterial species in pregnant women, Eur. J. Clin. Microbial. Infect. Dis. 1997, 6:517-22.
(13) Amsel, R., et al., Nonspecific vaginitis. Diagnostic criteria and microbial and epidemiologic associations, Am. J. Med 1983, 74:14-22;
(14) Larsson, P. G., Treatment for bacterial vaginosis: an update on the expected cure rate, Int'l J. of STD & AIDS 1997, 8:35-6.
(15) Larsson, P. G. Treatment of bacterial vaginosis. Int. J. STD AIDS 1992; 3: 239-247.
(16) Wilson, 2004, Sex Transm Infect 80:8-11.
(17) See, Hillier et al., June 1993, “Efficacy of Intravaginal 0.75% Metronidazole Gel for the Treatment of Bacterial Vaginosis,” Obstet Gynecol 81(6):963-967
(18) Scholes, D., et al., Vaginal douching as a risk factor for acute pelvic inflammatory disease, Obstet. Gynecol. 1993, 81:601-6;
(19) Darling J. R., et al., Vaginal douching and the risk of tubal pregnancy, Epidemiology 1991, 2:40-8;
(20) Kendrick, J. S., et al., Vaginal douching and the risk of ectopic pregnancy among black women, Am. J. Obstet. Gynecol. 1997, 176:991-7;
(21) Baird, D. D., et al., Vaginal Douching and reduced fertility, Am. J Public Health 1996, 86:844-50.
(22) Warr A J, et al. Sexually transmitted infections during pregnancy and subsequent risk of stillbirth and infant mortality in Kenya: a prospective study. BMJ 2018; 0:1-7. doi: 10.1136/sextrans-2018-053597
(23) Baqui et al. Prevalence of and risk factors for abnormal vaginal flora and its association with adverse pregnancy outcomes in a rural district in north-east Bangladesh Acta Obstet Gynecol Scand. 2018; 1-11.
(24) Bianchi-Jassir et al Preterm Birth Associated With GBS Maternal Colonization worldwide: Systematic review and meta—analyses. CID 2017:65 (Suppl 2)
(25) Lin F Y, Weisman L E, Troendle J, Adams K. Prematurity is the major risk factor for late-onset group B Streptococcus disease. J Infect Dis 2003; 188:267-71.
(26) Proc Natl Acad Sci USA. 2011; 108:4680-4687
(27) B. Vitali. C. Pugliese, E. Biagi, M. Candela, S. Turroni, G. Bellen, G. G. G. Donders, P. Brigidi, Dynamics of vaginal bacterial communities in women developing bacterial vaginosis, candidiasis, or no infection, analyzed by PCR-denaturing gradient gel electrophoresis and real-time PCR. Appl. Environ. Microb, 2020, 73(18), 5731-5741.
(28) V. D. Mijac, S. V. Dukic, N. Z. Opayski, M. K. Dukic, L. T. Ranin, Hydrogen peroxide producing lactobacilli in women with vaginal infections. Eur. J. Obstet. Gyn. R. B 2006, 129(1), 69-76.
(29) E. Biagi, B. Vitali, C. Pugliese, M, Candela, G. G. G. Donders, P. Brigidi, Quantitative variations in the vaginal bacterial population associated with asymptomatic infections: a real-time polymerase chain reaction study. Eur. J. Clin. Microbiol. 2009, 28, 281-285.
(30) L. Akimoto-Gunther, P. de Souza Bonfirm-Mendonca, G. Takahachi, M. M. T. Irie, S. Miyamoto, M. E. Lopes Consolaro, T. I. E. Svidzinsk, Highlights regarding host predisposing factors to recurrent vulvovaginal candidiasis: chronic stress and reduced antioxidant capacity. PLoS One 2016, 11(7), e0158870. DOI: 10.1371/journal. pone.0158870.
(31) C. Ceccarani, C. Foschi, C. Parolin, A. D'Antuono, V. Gaspari, C. Consolandi, L. Laghi, T. Camboni, B. Vitali, M. Severgnini, A. Marangoni, Diversity of vaginal microbiome and metabolome during genital infections. Sci. Rep. 2019, 9, 14095. DOI: 10.1038/s41598-019-50410-x.
(32) X. Zhou, R. Westman, R. Hickey, M. A. Hansmann, C. Kennedy, T. W. Osborn, L. J. Forney, Vaginal microbiota of women with frequent vulvovaginal candidiasis. Infect. Immun 2009, 77(9), 4130-4135. DOI: 10.1128/1A1.00436-09.
(33) Allonsius, C. N., Vandenheuvel, D., Oerlemans, E. F. M. et al. Inhibition of Candida albicans morphogenesis by chitinase from Lactobacillus rhamnosus GG. Sci Rep 9, 2900 (2019). https://doi.org/10.1038/s41598-019-39625-0.

1. A pharmaceutical composition for use in the prevention or treatment of a condition associated with a reduction in the number of Lactobacilli in the vagina of a subject relative to a healthy subject, wherein the composition comprises from 1.3 to 1.7 w/v % glycogen and less than 0.1 w/v % lactic acid.

2. A pharmaceutical composition according to claim 1 for the use according to claim 1 wherein the composition contains no lactic acid.

3. A pharmaceutical composition according to claim 1, further comprising water and a cellulose derivative, optionally wherein the composition comprises from 94 to 97 w/v % water and from 2 to 5 w/v % of a cellulose derivative.

4. A pharmaceutical composition according to claim 1, for the use according to any preceding claim wherein the composition comprises from 1.3 to 1.7 w/v % glycogen and an aqueous hydrogel comprising a cellulose derivative.

5. A pharmaceutical composition according to claim 1, for the use according to any preceding claim wherein the composition comprises from 1.4 to 1.6 w/v % glycogen, optionally about 1.5 w/v % glycogen.

6. A pharmaceutical composition according to claim 1, wherein the glycogen is obtainable from an animal source, optionally wherein the glycogen is oyster, mussel or bovine glycogen.

7. A pharmaceutical composition according to claim 3, for the use according to claim 3 wherein the cellulose derivative is a cellulose ether, optionally wherein the cellulose ether is methylcellulose (MC), hydroxypropyl methylcellulose (HPMC), ethyl cellulose (EC), hydroxyethyl cellulose (HEC), sodium carboxymethylcellulose (NaCMC), or combinations thereof.

8. A pharmaceutical composition according to claim 7 for the use according to claim 7 wherein the cellulose ether is hydroxypropyl methylcellulose.

9. A pharmaceutical composition according to claim 1 for the use according to claim 1 wherein the pH of the composition is from 5.7 to 6.7.

10. A pharmaceutical composition according to claim 1, for the use according to claim 1 which is in the form of a hydrogel.

11. A pharmaceutical composition according to claim 1 for the use according to claim 1 which is a vaginal pessary.

12. A pharmaceutical composition according to claim 1 for the use according to claim 1 which is mucoadhesive, optionally wherein the composition comprises a mucoadhesive film, gel or mucoadhesive in situ gelling liquid crystalline precursor system.

13. A pharmaceutical composition according to claim 1 further comprising one or more pharmaceutically acceptable excipients.

14. A pharmaceutical composition according to claim 1 for the use according to claim 1, further comprising one or more additional therapeutic agents.

15. A pharmaceutical composition according to claim 14 for the use according to claim 14, wherein the one or more additional therapeutic agents are selected from the group consisting of an antibiotic, an antibacterial agent, an antifungal agent, an anti-parasitic agent, an antiviral agent, antiseptic agent and an anti-inflammatory agent.

16. A method for the prevention or treatment of a condition associated with a reduction in the number of Lactobacilli in the vagina of a subject relative to a healthy subject, the method comprising the step of administering to a subject in need thereof a pharmaceutical composition as defined in any preceding claim.

17. A pharmaceutical composition for use or method according to claim 1, wherein the use or method comprises providing an increase in the number of Lactobacilli in the vagina of the subject relative to an untreated subject with the condition, optionally wherein the increase in the number of Lactobacilli is achieved for a period of at least 48 hours.

18. A pharmaceutical composition for use or method according to claim 1 wherein the use or method comprises restoring the number of Lactobacilli in the vagina to a normal level.

19. A pharmaceutical composition for use or method according to claim 1 wherein the use or method comprises attaining a normal pH of the vagina of the subject of from 3.8 to 4.5.

20. A pharmaceutical composition for use or method according to claim 1 wherein the condition to be prevented or treated is a vaginal infection, vaginal inflammation, or a complication therefrom.

21. A pharmaceutical composition for use or method according to claim 20, wherein the complication is one selected from the group consisting of preterm birth, stillbirth, postpartum vaginal infection and vaginal inflammation.

22. A pharmaceutical composition for use or method according to claim 1 wherein the condition to be prevented or treated is one selected from the group consisting of bacterial vaginosis, vaginitis, vulvovaginal candidiasis and Group B streptococcus infection.

23. A pharmaceutical composition for use or method according to claim 1 wherein the subject to be treated is pregnant.

24. A pharmaceutical composition for use or method according to claim 1 wherein the condition to be prevented or treated is associated with an increase in the number of pathogenic bacteria or yeast in the vagina of a subject relative to a healthy subject.

25. A pharmaceutical composition according to claim 1, any preceding claim for the use according to any preceding claim wherein the composition is suitable for intravaginal administration.

26. A pharmaceutical composition according to claim 9 wherein the pH of the composition is from 6.0 to 6.2.

27. A pharmaceutical composition for use or method according to claim 20 wherein the condition to be prevented or treated is a recurrent vaginal infection or inflammation.

28. A pharmaceutical composition for use or method according to claim 24 wherein the condition to be prevented or treated is one sected from the group consisting of Gardnella vaginalis or Group B Streptococcus bacteria and a species of Candida yeast, optionally Candida albicans.
